(19) United States
(12) Patent Application Publication
(10) Pub. No.: US 2022/0372434 A1
(43) Pub. Date: Nov. 24, 2022

(54) A system and process for producing a biosurfactant and formulations comprising same

(71) Applicant: Global Bioprotect IP Pty Ltd, Port Melbourne, Victoria (AU)

(72) Inventors: Paul SMYTH, Port Melbourne, Victoria (AU); Gary WILLETT, Port Melbourne, Victoria (AU); Stacey ROBERTS, Port Melbourne, Victoria (AU)

(21) Appl. No.: 17/766,050

(22) PCT Filed: Oct. 2, 2020
(86) PCT No.: PCT/AU2020/051066

§ 371 Date: Apr. 1, 2022

(30) Foreign Application Priority Data
Oct. 4, 2019 (AU) 2019903737

(51) Int. Cl. C12N 1/20; C12M 1/12; C12M 3/00; C12M 1/00; C12M 1/06; C12M 1/34; C11D 3/48; C11D 3/20; C11D 3/00

(52) U.S. Cl. CPC C12N 1/205; C12M 23/06; C12M 23/44; C12M 23/58; C12M 27/02; C12M 27/20; C12M 29/18; C12M 41/04; C12M 47/10; C11D 3/48; C11D 3/2034; C11D 3/2086; C11D 3/0047; C12R 2001/125

(57) ABSTRACT

The present invention generally relates to a system and process for producing a biosurfactant, and more particularly to a system and process for producing a biosurfactant from at least one strain of Bacillus subtilis, and formulations which comprise the biosurfactant.

### TECHNICAL FIELD

The present invention generally relates to a system and process for producing a biosurfactant, and more particularly to a system and process for producing a biosurfactant from at least one strain of Bacillus subtilis, and formulations which comprise the biosurfactant.

### BACKGROUND

There has been significant interest in the production of biosurfactants, such as surfactin, as they are very powerful surfactants. Surfactants are compounds that are able to alter the interfacial tension of liquids at very low concentrations. A biosurfactant is a surfactant that is produced by living cells, such as microorganisms. Many biosurfactants have antimicrobial activity and other useful properties. The following paragraphs describe such biosurfactants in further detail.

Cyclic lipopeptides, such as surfactin, have a cyclic peptide moiety and a moiety derived from a fatty acid. Surfactin has a cyclic peptide of seven amino acids including both D- and L-amino acids, Glu-Leu-D-Leu-Val-Asp-D-Leu-Leu, linked from the N-terminus to the C-terminus to form a cyclic moiety by a C12-C17 β-hydroxy fatty acid as shown below.

Lichenysin has a similar structure with the amino acid sequence differing from surfactin. Gln-Leu-D-Leu-Val-Asp-D-Leu-Ile, linked from the N-terminus to the C-terminus to form a cyclic moiety by a C12-C17 β-hydroxy fatty acid.

Fengycin is a cyclic lipopeptide having the sequence Glu-D-Orn-Tyr-D-Allo-Thr-Glu-D-Ala-Pro-Glu-D-Tyr-Ile where the peptide is cyclized between the tyrosine phenoxy group of position 3 and the C-terminus of the Ile at position 10, the fatty acid is attached to the peptide forming an amide with the N-terminus.

Iturin refers to a group of cyclic peptides with the sequence Asn-D-Tyr-D-Asn-Gln-Pro-D-Asn-Ser in which the N-terminus and C-terminus are connected by a β-amino fatty acid of varying length.

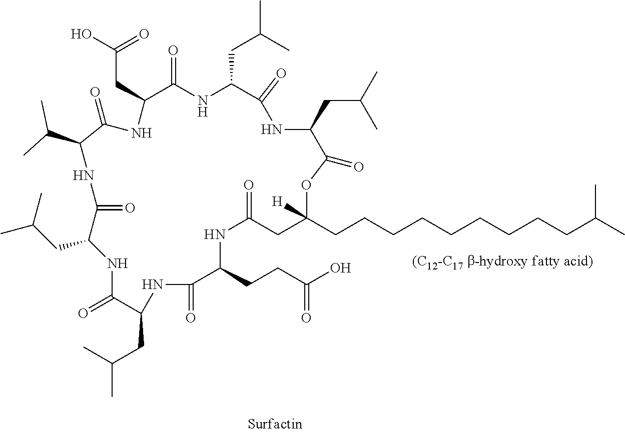 [SMILES: CC(C)CCCCCCCCC[C@@H]1CC(=O)N[C@@H](CCC(=O)O)C(=O)N[C@@H](CC(C)C)C(=O)N[C@H](CC(C)C)C(=O)N[C@@H](C(C)C)C(=O)N[C@@H](CC(=O)O)C(=O)N[C@H](CC(C)C)C(=O)N[C@@H](CC(C)C)C(=O)O1]

Such biosurfactants may be produced through bacterial activity. Current methods of producing these biosurfactants have focused on the identification of high yielding strains of Bacillus subtilis [U.S. Pat. No. 3,030,789, Mulligan et al., 1989, Applied Microbiology and Biotechnology 31:486-489], or by adjusting culture conditions such as culturing in a magnetic field [JP-A-6-121 668], high iron concentrations [Wei et al., Enz. Microbial. Technol. 1989, 22:724-728], in the presence of peat [Sheppard et al. 1989, Appl. Microbial. Biotechnol. 27:486-489] or reduced oxygen [Kim et al., J. Fermenl. Bioeng. 1997, 84:41-46].

However, the use of such methods do not yield sufficiently high concentrations the biosurfactants to make such methods economically viable for industrially scaled production. Further such methods commonly rely on high cost ingredients or apparatuses.

The preferred embodiments of the present invention seek to address one or more of these disadvantages, and/or to at least provide the public with a useful alternative. The present invention allows for a commercially viable high yielding process to produce such biosurfactants and as such also provides new and useful disinfectant formulations as a result.

Further, the risk of contamination requires the addition of preservatives or other surfactants to control the growth of complex organisms, such as mould.

The reference in this specification to any prior publication (or information derived from the prior publication), or to any matter which is known, is not, and should not be taken as an acknowledgment or admission or any form of suggestion that the prior publication (or information derived from the prior publication) or known matter forms part of the common general knowledge in the field of endeavour to which this specification relates.

### SUMMARY

In a first aspect, there is provided a system for producing a biosurfactant from at least one strain of Bacillus subtilis, the system comprising: at least one reactor arranged to receive a mixture of foamate and aqueous biosurfactant cultured from the at least one strain of Bacillus subtilis, the at least one reactor being arranged to ferment the mixture of foamate and aqueous biosurfactant into a concentrated foamate of biosurfactant, the at least one reactor comprising: an oxygenation module arranged to oxygenate the mixture of foamate and aqueous biosurfactant; an agitation module arranged to agitate the mixture of foamate and aqueous biosurfactant; a recirculation module arranged to recirculate the mixture of foamate and aqueous biosurfactant; and the system further including a collection module in connection with the at least one reactor that is arranged to continuously collect the concentrated foamate of biosurfactant.

In an embodiment, the at least one reactor is a cylindrical vessel, with a base, a top and a side wall.

In an embodiment, the at least one reactor has a height to a diameter ratio of 3:1.

In an embodiment, the recirculation module includes: a draining pipe in connection with at least one drain formed in the base or the side wall of the at least one reactor; a pump arranged to pump the mixture of foamate and aqueous biosurfactant drained into the draining pipe; a recirculation tank in connection with the draining pipe, the recirculation tank arranged to include at least one recirculation air diffuser to oxygenate the mixture of foamate and aqueous biosurfactant in the recirculation tank; and a recirculation pipe arranged reintroduce the mixture of foamate and aqueous biosurfactant from the recirculation tank into the at least one reactor.

In an embodiment, the agitation module includes at least one agitation mechanism arranged to generate a flow path in the mixture of foamate and aqueous biosurfactant.

In an embodiment, the at least one agitation mechanism includes an impeller connected to a rotation shaft located at the base of the at least one reactor.

In an embodiment, the agitation module further includes at least one baffle arranged to extend at least partially along the side of wall between the base and the top.

In an embodiment, the flow path is a substantially circular path around a circumference of the at least one reactor.

In an embodiment, the flow path is a substantially conical helix shape within the at least one reactor.

In an embodiment, the concentration of the concentrated foamate of biosurfactant is in the range of 50 to 2000 ppm.

In an embodiment, the oxygenation module includes at least one air diffuser arranged within the at least one reactor to supply air to the mixture of foamate and aqueous biosurfactant.

In an embodiment, the air includes a mix of pure oxygen and pressurised air.

In an embodiment, the mix of pure oxygen and pressurised air has an oxygen concentration of approximately 40%.

In an embodiment, the mix of pure oxygen and pressurised air is mixed in a mixing tank in connection with an air compressor and an oxygen generator.

In an embodiment, the at least one reactor includes an additive module.

In an embodiment, the additive module is configured to allow for the addition of at least one additive to the at least one reactor whilst maintaining the system in a closed state.

In an embodiment, the at least one additive includes one or more items selected from the group of; monopotassium phosphate, disodium phosphate, magnesium sulphate, calcium chloride, ethylenediaminetetraacetic acid, ferrous sulphate, manganese sulphate, dextrose anhydrous, yeast extract, ammonium chloride, sodium nitrate.

In an embodiment, the at least one additive is added to the at least one reactor at one or more times during the third to fourth hour since commencement of fermentation of the mixture of foamate and aqueous biosurfactant.

In an embodiment, the at least one additive may further include a caustic solution, which is added to maintain the mixture of foamate and aqueous biosurfactant at a pH level between 6.9 and 7.3.

In an embodiment, the system further includes a pH maintaining device that is arranged to regularly detect the pH and add an appropriate amount of the caustic solution to maintain the mixture of foamate and aqueous biosurfactant at a pH level between 6.9 and 7.3.

In an embodiment, the collection module includes at least one foamate stack arranged to connect to the at least one reactor at a first end of the foamate stack.

In an embodiment, the collection module includes a foamate collection tank arranged to connect to a second end of the at least one foamate stack.

In an embodiment, the air provided to the at least one reactor by the one or more oxygenation modules flows up the at least one foamate stack and urges the concentrated foamate of biosurfactant into the foamate collection tank.

In an embodiment, the foamate collection tank is sized to enable continuous collection of the concentrated foamate of biosurfactant.

In an embodiment, the foamate collection tank includes a vent to exhaust the air.

In an embodiment, the system further includes a purified water module arranged to supply sterilized deionized water to the at least one reactor.

In an embodiment, the purified water module includes a heating module to heat the water from the water supply to a temperature between 40 to 50 degrees Celsius.

In an embodiment, the purified water module includes a filtration module to filter water from a water supply.

In an embodiment, the purified water module includes a sterilisation module to sterilise water from a water supply.

In an embodiment, the system further includes a temperature monitoring system that is arranged to automatically monitor and maintain the temperature mixture of foamate and aqueous biosurfactant within the at least one reactor within a range of 40 to 50 degrees Celsius.

In a second aspect, there is provided a process for producing a biosurfactant from at least one strain of Bacillus subtilis, the process comprising the following steps: producing a mixture of foamate and aqueous biosurfactant cultured from the at least one strain of Bacillus subtilis; fermenting the mixture of foamate and aqueous biosurfactant in at least one reactor to produce a concentrated foamate of biosurfactant, the at least one reactor arranged to: oxygenate the mixture of foamate and aqueous biosurfactant using an oxygenation module; agitate the mixture of foamate and aqueous biosurfactant using an agitation module; and recirculate the mixture of foamate and aqueous biosurfactant using a recirculation module; wherein the process further comprises the step of continuously collecting the concentrated foamate of biosurfactant from the at least one reactor using a collection module.

In a third aspect, there is provided an aqueous disinfectant formulation comprising:
(a) a biosurfactant composition comprising Surfactin, Iturin, Fengycin, and sodium dodecylbenzene sulfonate (DDBSA), wherein the Surfactin is present within the composition at between about 25-75 ppm,
(b) thymol;
(c) lactic acid; and
(d) preservative;
   wherein the formulation has a pH in the range of about 2.5-4.5.

### BRIEF DESCRIPTION OF FIGURES

Example embodiments are apparent from the following description, which is given by way of example only, of at least one non-limiting embodiment, described in connection with the accompanying figures.

FIG. 1 illustrates a process flow of an embodiment of the invention.

FIG. 2 illustrates a process flow of an embodiment of the invention.

FIG. 3 illustrates an example of an embodiment of the invention.

### DETAILED DESCRIPTION

The following modes, given by way of example only, are described in order to provide a more precise understanding of one or more embodiments. In the figures, like reference numerals are used to identify like parts throughout the figures.

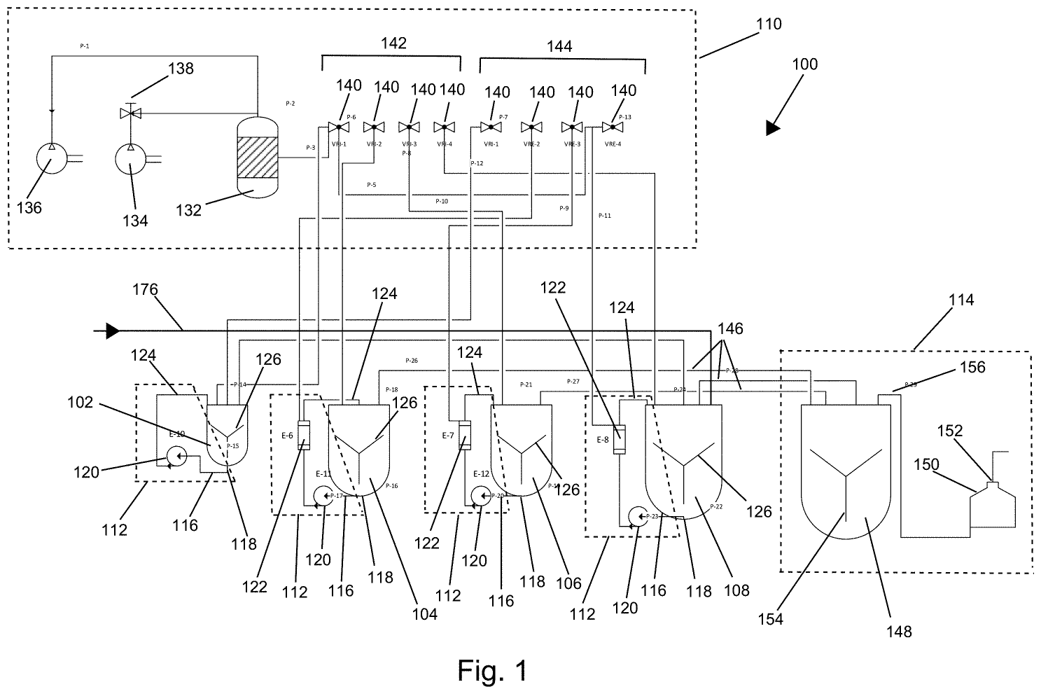

With general reference to FIGS. 1 to 3, the invention is described in relation to a system 100 for producing a biosurfactant from at least one strain of Bacillus subtilis. The system 100 comprises at least one reactor arranged to receive a mixture of foamate and aqueous biosurfactant cultured from the at least one strain of Bacillus subtilis.

The present system has been specifically designed for specific strains of Bacillus subtilis which produce improved yields of biosurfactants such as B. subtilis ATCC 21331, B. subtilis ATCC 21332, B. subtilis SD901 (FERM BP.7666). Many strains of biosurfactant-producing microbes are commercially or publicly available. In particular embodiments, the at least one strain of B. subtilis NRRL B-3383 or B. subtilis ATCC 21331 both of which are publicly available. In other embodiments, the biosurfactant-producing microorganism is B. subtilis RSA-203, a new strain of B. subtilis found to produce significant yields of the biosurfactant, surfactin, deposited with the ATCC on 9 Jan. 2013 under Accession No. PTA-13451, which is particularly preferred.

The mixture of foamate and aqueous biosurfactant may be initially cultured in a laboratory environment by forming an inoculum. The inoculum may be formed by adding deionized (DI) water and yeast extract to a flask in the approximate ratio of 2:3 and agitating these constituents until the contents of the flask is fully mixed. Once fully mixed, the yeast extract and water mixture is autoclaved, which kills any active yeast. The autoclaved yeast extract provides a food source for the at least one strain of Bacillus subtilis.

Once autoclaved, the at least one strain of Bacillus subtilis and various mineral salts are added to a portion of the yeast water mixture, where the remainder of the yeast extract water mixture is set aside. The mineral salts may include additives, such as but not limited to, monopotassium phosphate, disodium phosphate, magnesium sulphate, calcium chloride, ethylenediaminetetraacetic acid, ferrous sulphate, manganese sulphate, sodium nitrate and ammonium chloride. The phosphates, sulphates and chlorides etc. are trace elements to encourage growth and buffer the pH. This process forms an inoculum mixture, which then may be incubated for a period of time at a temperature of 36 degrees Celsius (° C.) whilst being agitated. For example, the inoculum mixture may be incubated on shaker tables operated at approximately 150 revolutions per minute (rpm). The inoculum mixture is incubated until it reaches an optical density measurement reading at 660 nm of 1.3 to 1.4 nm. The inoculum mixture forms a mixture of foamate and aqueous biosurfactant. That is, the mixture is a fluid solution with a small amount of foamate on top.

The at least one reactor may be arranged to ferment the mixture of foamate and aqueous biosurfactant into a concentrated foamate of biosurfactant. The highest concentrations of the biosurfactant are found in the foamate that is produced during fermentation. Producing the biosurfactant at high concentrations is advantageous for numerous reasons described later in the specification.

The system 100 may include multiple reactors, where the reactors may be the same size or be of different sizes. The at least one reactor may be made from a non-reactive metal, such as 316 stainless steel. In an example, the system 100 may include four reactors, namely a starter reactor 102, a first reactor 104, a second reactor 106 and a third reactor 108. In the example provided, the starter reactor 102 is the smallest vessel and the third reactor 108 is the largest vessel. Alternatively, the first 104, second 106 and third 108 reactors vessels may be the same size. In a further alterative embodiment, additional or less reactors than the example may also be used.

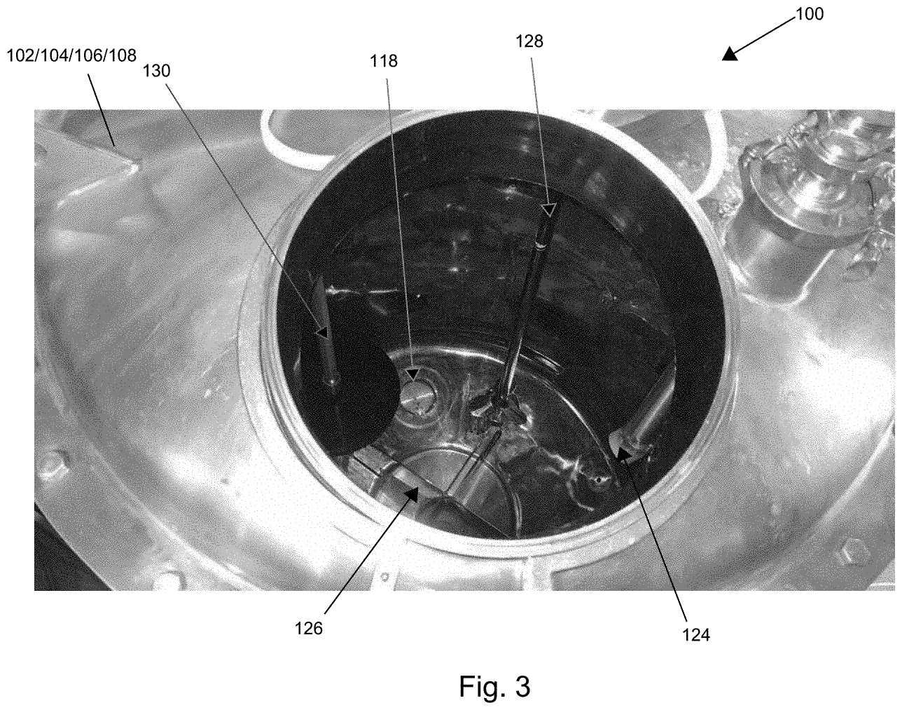

Referring specifically to FIG. 3, an example of the at least one reactor is provided. When referring to features that are common to each of the starter reactor 102, the first reactor 104, the second reactor 106 and/or the third reactor 108, the term “the reactors” may be used to collectively refer to the starter reactor 102, the first reactor 104, the second reactor 106 and the third reactor 108 unless otherwise stated. FIG. 3 illustrates an example interior arrangement of the reactors. The reactors may be made from stainless steel cylindrical vessel, each with a base, a top and a side wall.

Further, the reactors may each have a specific geometry to aid the fermentation process. For example, the reactors may have a height to a diameter ratio of 3:1; as such a ratio provides a sufficient surface-area interface between air at the top of each of the reactors and the mixture of foamate and aqueous biosurfactant within each of the reactors. Providing an ideal surface-area interface enhances the transformation of the bubbles from the oxygenation module into the foamate, which aids in concentrated foamate production.

In light of the variations above, the skilled addressee would understand that the number of reactors, the size, and the height relative to diameter dimensions may vary or be identical between the reactors of the system 100. Therefore, the skilled addressee would understand that such variation would be considered to be within the scope of the invention as described and defined herein.

In an example, the fermenting process may begin in the starter reactor 102, which receives a mixture of the aforementioned mineral salts, dextrose anhydrous, the remainder of the yeast extract water and warm water. The warm water may be sterilized deionized water (DI water) heated to a temperature of 42 Celsius (° C.), where the module for this process is described later in the description.

The mixture is mixed until fully combined and the inoculum mixture is added. The resulting mixture includes the mixture of foamate and aqueous biosurfactant and the additives to feed and encourage the growth of the at least one strain of Bacillus subtilis during fermentation of the resulting mixture. Furthermore, the starter reactor 102 may include other features to encourage growth of the at least one strain of Bacillus subtilis. For example, the starter reactor 102 may include a diffuser disk and diffuser ball to sparge the resulting mixture during fermentation.

The resulting mixture is fermented in the starter reactor 102 until the resulting mixture reaches sufficient bacterial cell density. Cell density refers to the number of cells per unit of volume which indicates the concentration of the at least one strain of Bacillus subtilis within the resulting mixture. For example, samples may be taken periodically from the resulting mixture and tested using conventional optical density measurement techniques to determine an optical density measurement at a point in time. This sampling process may be repeated until such time as the resulting mixture reaches an optical density measurement of 1.3 at 660 nm or higher.

Once sufficient amounts of the at least one strain of Bacillus subtilis have been produced, the resulting mixture is distributed into the first 104, second 106 and third 108 reactors to ferment of the mixture of foamate and aqueous biosurfactant. Using a starter reactor 102 to grow the at least one strain of Bacillus subtilis ensures that there is sufficient bacterial mass or cell density to seed the other reactors. In other words, the system 100 may include a two-step process, wherein the starter reactor 102 ferments the resulting mixture to increase the concentration of the at least one strain of Bacillus subtilis and the first 104, second 106 and third 108 reactors ferment the mixture of foamate and aqueous biosurfactant to form a concentrated foamate of biosurfactant. In an embodiment, the resulting mixture may be first transferred to the largest third reactor 108 before later sharing the resulting mixture between the remaining two reactors in order to stagger the fermentation process and speed up the overall fermenting time.

In an embodiment, each of the reactors may include a number of modules. In use, the system conditions provided and/or maintained by each of the modules synergistically increases the concentration of the foamate of biosurfactant produced during the fermentation of the mixture of foamate and aqueous biosurfactant. Each of the reactors may comprise an oxygenation module 110 arranged to oxygenate the mixture of foamate and aqueous biosurfactant, an agitation module arranged to agitate the mixture of foamate and aqueous biosurfactant, and a recirculation module 112 arranged to recirculate the mixture of foamate and aqueous biosurfactant. The system 100 may further include a collection module 114 in connection with the at least one reactor that is arranged to continuously collect the concentrated foamate of biosurfactant. Each of these modules is described in further detail later in the specification.

During the fermentation of the mixture of foamate and aqueous biosurfactant within the first 104, second 106 and third 108 reactors, the oxygenation module 110, the agitation module, and the recirculation module 112 are arranged to encourage foamate production. In other words, the reactors ferment the mixture of foamate and aqueous biosurfactant into a concentrated form of the biosurfactant, wherein the highest concentrations of the biosurfactant are in the foamate. For example, the concentration of the concentrated foamate of biosurfactant may be in the range of 50 to 2000 ppm when it is collected. During the fermentation process, the concentrated foamate of biosurfactant rises to the top of the first 104, second 106 and third 108 reactors and is collected by the collection module 114.

Foam is the dispersion of particles (gas) in continuous medium (liquid). Examples of foam are soap, beer, fire extinguisher, etc. Continuous foam fractionation occurs by generating bubbles when aerating or sparging gas into a liquid solution. The biosurfactants (surface-active molecules) are adsorbed on the surface of the bubbles. When the foam is formed, it is collected and collapsed to make foamate, rich in biosurfactants.

A protein molecule is an amphiphilic molecule, which has a hydrophilic region (part that ‘loves’ water) and a hydrophobic region (part that ‘fears’ water) simultaneously. This property classified the molecule as a biosurfactant. The affinity of a protein molecule for the surface tends to be high due to the interactions of many hydrophobic force-driven points of attachments to the interface. Hence, the hydrophobic region of a protein molecule is naturally stronger than its hydrophilic region, which results in the molecule having large and positive surface excess.

Therefore, protein molecules will readily adsorb and form a monolayer on the gas-liquid interfaces (either the surface of bubbles or solution). The surface tension of the surface of the bubbles prevents the adsorbed protein molecules from escaping. As more bubbles are formed, they will merge and form foam on the surface of the solution when sufficiently stable.

The extent of adsorption of the protein molecules on the gas-liquid interface is governed by the Gibbs adsorption equation. In this equation, surface excess is the number of molecules adsorbed on the interface while chemical potential is the ability of a molecule in a system to perform work. During the adsorption process, work is done by the molecule to adsorb on the interface, which leads to increase in chemical potential. Therefore, the presence of biosurfactants decreases the surface tension of the solution to ease the extraction of the biosurfactants. Gibbs adsorption isotherm is used when thermal equilibrium is established (at constant temperature).

In light of the above, the system 100 includes a number of modules that are arranged to optimise the production of foamate. The various modules of the system 100 are now discussed in further detail. With reference to FIGS. 1 and 3, an embodiment is provided, where the recirculation module 112 provided to the reactors may include a draining pipe 116 in connection with a drain 118 formed in a base or side of the reactors. The recirculation module 112 may further include a pump 120 arranged to pump the mixture of foamate and aqueous biosurfactant drained into the draining pipe 116. The pump 120 may be a diaphragm pump. Diaphragm pumps may provide reduced shear in comparison to other types of pumps, where shear is defined as relative motion between adjacent layers of a moving fluid. Further, diaphragm pumps also provide more variability of pumping speeds by reducing or increasing airflow to the pump. However, as would be understood by a person skilled in the art, other low shear and variable speed pump types may be used.

The recirculation module 112 may also include a recirculation tank 122 in connection with the pump 120, wherein the recirculation tank 122 is arranged to include at least one recirculation air diffuser (not shown) to oxygenate the mixture of foamate and aqueous biosurfactant in the recirculation tank 122. In one embodiment, the starter reactor 102 may include the recirculation module 112 without the recirculation tank 122. In another embodiment, first reactor 104, second reactor 106 and third reactor 108 may include the recirculation module 112 with the recirculation tank 122.

Moreover, the recirculation module 112 may further include a recirculation pipe 124 arranged reintroduce the mixture of foamate and aqueous biosurfactant from the recirculation tank 122 into the reactors. The recirculation module 112 enables oxygenation of the mixture of foamate and aqueous biosurfactant within a loop, the loop being arranged external to the reactors. The oxygenation of the mixture of foamate and aqueous biosurfactant aids in foamate production, as discussed further below. Further, the movement of the mixture of foamate and aqueous biosurfactant from the reactors, through the recirculation module 112 and back into the reactors aids in agitating the mixture of foamate and aqueous biosurfactant, which also aids in concentrated foamate production.

In an embodiment, the agitation module may include at least one agitation mechanism 126 arranged to generate a flow path in the mixture of foamate and aqueous biosurfactant. For example, the at least one agitation mechanism 126 may include an impeller connected to a rotation shaft located at the base of each of the reactors. The rotation shaft may connected to a motor, the motor being arranged to rotate the shaft and impeller. The impeller blades may be of varying sizes, shapes and quantity. For example, as shown in FIG. 3, the impeller includes four flat blades symmetrically disposed around the shaft (not shown). The speed at which the at least one agitation mechanism 126 is rotated may also vary depending on the number of blades, the blade surface area and the blade shape. For example, the at least one agitation mechanism 126 shown in FIG. 3 may be rotated at approximately 150 rpm although it would be understood by the skilled addressed that other speeds may be used. The rotation speed of the at least one agitation mechanism 126 is set to such a speed so to enable the generation of the flow path in the mixture of foamate and aqueous biosurfactant. This agitation aids in concentrated foamate production. However, the speed of the at least one agitation mechanism 126 must also be limited so as to reduce any shear forces experienced by the mixture of foamate and aqueous biosurfactant during agitation, as high amounts of shear is harmful to the bacteria, and hence hinders concentrated foamate production.

During fermentation, the concentrated foamate of biosurfactant floats to the air-fluid interface at the top of the mixture of foamate and aqueous biosurfactant. In one embodiment, the flow path may be a substantially circular path around a circumference of the reactors. That is, the flow path within the plane of the air-fluid interface may be substantially circularly shaped. Alternatively, the flow path may be a conic helical shape within the at least one reactor. In other words, the flow path in the three-dimensional space defined by the reactors may be a substantially conical helix shape expanding outwards from the at least one agitation mechanism 126.

Due to the centrifugal force experienced by the concentrated foamate of biosurfactant as it follows the flow path, the concentrated foamate of biosurfactant is pushed towards the sides of the reactors. As more of the concentrated foamate of biosurfactant is produced, the concentrated foamate of biosurfactant begins to climb the sides of the reactors towards the top of the reactor. This process is synergistically aided by the function of the oxygenation module 110, which is discussed further below.

Further, the agitation module may also include at least one baffle 128 arranged to extend at least partially along the side of wall between the base and the top of the reactors. The at least one baffle 128 may include a flow-directing or obstructing vane or panel that is arranged inside the reactors to direct the flow of fluids for maximum efficiency. As such, the at least one baffle 128 may be arranged to improve the mixing of the various components of the mixture of foamate and aqueous biosurfactant to aid in concentrated foamate production.

In a further embodiment, the oxygenation module 110 may include a number of sub-modules that are arranged to oxygenate the mixture of foamate and aqueous biosurfactant. Within the context of the description, the term oxygenate is taken to mean the introduction of air to the mixture of foamate and aqueous biosurfactant, wherein the air includes oxygen and other gasses that support the growth of the at least one strain of Bacillus subtilis, not just oxygen. In other words, the term oxygenate is understood to mean aerate.

With continued reference to FIGS. 1 and 3, an embodiment is provided wherein the oxygenation module 110 includes at least one air diffuser 130 arranged to locate within the reactors, wherein the at least one air diffuser 130 supplies air to the mixture of foamate and aqueous biosurfactant. The at least one air diffuser 130, also referred to as a membrane diffuser, is an aeration device typically in the shape of a disc, tube or plate, which is used to transfer air into a fluid. Air diffusers may use either rubber membranes or ceramic elements typically and produce either fine or coarse bubbles. Further, the above mentioned surface-area of the air-fluid interface from a reactor with a height to a diameter ratio of 3:1 as described above enhances the transformation of the bubbles from the oxygenation module into the foamate, which synergistically aids in concentrated foamate production.

The air may include a mix of pure oxygen and pressurised air. For example, the mix of pure oxygen and pressurised air may have an oxygen concentration of approximately 40%. The mix of pure oxygen and pressurised air may be mixed in a mixing tank 132 in connection with an air compressor 134 and an oxygen generator 136. The oxygen generated by the oxygen generator 136 is at approximately 94% to 98% and the flow of the compressed air from the air compressor 134 is regulated by a regulator valve 138, for example such as a screw down valve.

The air flow from the mixing tank 132 is regulated via a plurality of sparge valves 140, where each sparge valve 140 is arranged to connect with the either one of the reactors or one of the recirculation modules provided to one of the reactors. As such, the plurality of sparge valves 140 are categorised into two groups, internal sparge valves 142 and external sparge valves 144. Alternatively, an embodiment is provided wherein the air flow from the mixing tank 132 is regulated by multiple air ports in connection with the mixing tank 132.

In an embodiment, the first 104, second 106 and third 108 reactors may include an additive module (not shown). The additive module may be configured to allow for the addition of at least one additive to the reactors whilst maintaining the system in a closed state. In other words, the additive module may be configured to allow additional additives to be added to the reactors during fermentation without opening the reactors and disturbing the balance of the system. Disturbing the balance of the system may allow the ingress of contaminants and disrupt the air flow produced by the oxygenation module 110, which has the effect of hindering concentrated foamate production. As such, the additive module enables for additional additives to be added to the system 100 to encourage the growth and activity of the at least one strain of Bacillus subtilis without compromising the system 100.

For example, the additive module may be arranged to provide additional additives, such as yeast and/or mineral salts, to the reactors during fermentation. The additive module may be arranged to connect in series with the recirculation module 112. The additive module may include a “T” shaped section of piping including valves located at the suction side of the pump 120. To add the additional additives, the suction from the bottom of the reactor is temporary closed and the valve to the additional additive supply is opened to mix the additives into the mixture of foamate and aqueous biosurfactant. The additional additives may be contained within a chemical storage tote, which is connected to the “T” shaped section of piping. The additional additives pass through the recirculation module 112 then into the top of the reactors. Once the supply of additional additives is exhausted, the valves are reopened and the normal recirculation process continues. Alternatively, the additive module may be connected to the reactors via a separate pipe that provides the additional additives to the reactors independent of the recirculation module 112.

As such, the additives may include the yeast extract, mineral salts and/or dextrose used to culture the at least one strain of Bacillus subtilis and added at the beginning of the fermentation process. As such, the at least one additive may include one or more items selected from the group of; monopotassium phosphate, disodium phosphate, magnesium sulphate, calcium chloride, ethylenediaminetetraacetic acid, ferrous sulphate, manganese sulphate, dextrose anhydrous, yeast extract, ammonium chloride, sodium nitrate.

The growth of the at least one strain of Bacillus subtilis during fermentation may be modelled by a constant growth gradient. For example, the population of the at least one strain of Bacillus subtilis may double every twenty minutes during the third and fourth hour of fermentation, where the fermentation process may take up to as many as 7 hours. Due to this explosive growth, the system 100 may be configured to support that growth by adding the at least one additive, such as yeast extract, to the reactors at one or more times during the third to fourth hour since commencement of fermentation of the mixture of foamate and aqueous biosurfactant. The provision of additional additives during the explosive growth phase aids the at least one strain of Bacillus subtilis in producing the concentrated foamate, without compromising the system 100 where compromising the system 100 would hinder concentrated foamate production.

In another embodiment, the at least one additive may further include a caustic solution, which is added to maintain the mixture of foamate and aqueous biosurfactant at a pH level between 6.9 and 7.3. As the at least one strain of Bacillus subtilis produces the concentrated foamate of biosurfactant, the bacteria also produces lactic acid (from breakdown of glucose) that in turn lowers the pH of the mixture of foamate and aqueous biosurfactant. If the pH level of the mixture of foamate and aqueous biosurfactant drops below 6.9, the activity of at least one strain of Bacillus subtilis may cease. As such, it is important to adjust the pH of the mixture of foamate and aqueous biosurfactant during fermentation. For example, in one embodiment, the system 100 may further includes a pH maintaining device (not shown) that is arranged to regularly detect the pH of the mixture of foamate and aqueous biosurfactant and add an appropriate amount of the caustic solution to the mixture of foamate and aqueous biosurfactant to, for example, maintain the mixture of foamate and aqueous biosurfactant at a pH level between 6.9 and 7.3.

For example, a pH maintaining device may be provided to each of the reactors, each of the pH maintaining devices periodically samples the mixture of foamate and aqueous biosurfactant in each reactor and adds the caustic solution, such as sodium hydroxide at 25-50% concentration, to each reactor to maintain the pH above 6.8. For example, the pH may be maintained in the range of 7 to 7.3.

With continued reference to FIG. 1, an embodiment is provided wherein the collection module 114 includes at least one foamate stack 146, wherein the at least one foamate stack 146 includes a first end and a second end, wherein each of the first 104, second 106 and third 108 reactors are connected to the first end of at least one foamate stack 146. In other words, each of the first 104, second 106 and third 108 reactors may be connected to the collection module 114 by its own foamate stack 146, the foamate stacks 146 being arranged to collect the concentrated foamate of biosurfactant. The at least one foamate stack 146 may be a tube of sufficient diameter, which protrudes in a substantially vertical manner from the top of the reactors. For example, tube diameter may be in the range of 10 centimetres to 20 centimetres. The second end of the at least one foamate stack 146 may be connected a foamate collection tank 148 via a tube or pipe. The foamate collection tank 148 may include an overflow tank with a vent 152 for excess air. The vent 152 may include a unidirectional valve to ensure that air is vented without the ingress of air or contaminants.

As mentioned above, the agitation of the mixture of foamate and aqueous biosurfactant during fermentation by the agitation module pushes the concentrated foamate of biosurfactant towards the sides of the first 104, second 106 and third 108 reactors. As the at least one strain of Bacillus subtilis produces more and more concentrated foamate of biosurfactant, the foamate is pushed up against the walls and begins to move up or “climb” the side wall of the reactors.

The “climbing action” is aided by the flow of air provided to the at least one reactor by the oxygenation module 110. That is, the oxygenation module 110 is arranged to supply air to each of the reactors, with the only outlet for that air being the at least one foamate stack 146. The air provided by the oxygenation module 110 provides an air flow path that flows up the at least one foamate stack 146, and in doing so “urges” the concentrated foamate of biosurfactant into the foamate collection tank 148. The term “urges” describes moving the concentrated foamate without another object directly contacting the concentrated foamate. The biosurfactants are attached to the foamate, it is important that the foam bubbles in the foamate do not burst prematurely. Hence, it is important for the concentrated foamate of biosurfactant to be collected efficiently and gently. As such, by the cooperative effects of the oxygenation module 110 and the agitation module achieve the gentle collection of the concentrated foamate of biosurfactant from the first 104, second 106 and third 108 reactors.

The air flow path continues through the foamate collection tank 148 and into at least one holding tank 150, where the air leaves the system 100 through vent 152 provided to the holding tank 150. The holding tank 150 may also be used to temporarily retain the concentrated foamate of biosurfactant before final product processing. Alternatively, an additional holding tank 150 may also be included and used as an overflow collection tank.

In an embodiment, the foamate collection tank 148 may be sized to enable continuous collection of the concentrated foamate of biosurfactant. That is, the foamate collection tank 148 may be sufficiently large to enable the concentrated foamate of biosurfactant continuously from the reactors during fermentation. For example, the foamate collection tank 148 may be 4000 litres up to 8000 L. Continuous removal of the biosurfactant creates a stimulus for the Bacillus subtilis to increase production of biosurfactant in response to the lowering concentration of the biosurfactant in the tank environment. Therefore, the arrangement of a sufficiently large foamate collection tank 148 aids in concentrated foamate production.

Furthermore, in an embodiment, the foamate collection tank 148 may include a collection tank agitator 154 that is arranged to agitate the concentrated foamate of biosurfactant. In a similar manner to the agitator mechanisms 126 in each of the reactors, the collection tank agitator 154 may be arranged to agitate the the concentrated foamate of biosurfactant. Agitation of the concentrated foamate in the foamate collection tank has a dual purpose. Firstly, the collection tank agitator 154 blends the foamate collected from each of the reactors. Blending in this manner averages the concentration of the concentrated foamate of biosurfactant and the pH of the concentrated foamate of biosurfactant to provide a more consistent product to the end consumer. Secondly, agitating the concentrated foamate in this manner results in the concentrated foamate of biosurfactant being pushed towards the side of the foamate collection tank 148. As more of the concentrated foamate of biosurfactant is collected, the concentrated foamate of biosurfactant in the foamate collection tank 148 begins to climb the sides of the tank 148. This process is also aided by the function of the oxygenation module 110 in a similar manner to what is described above. The concentrated foamate of biosurfactant is passed upwards through a tube 156 and into the holding tank 150 in a similar manner to how the concentrated foamate of biosurfactant is passed through the foamate stack 148 and into the foamate collection tank 148.

The system may further include a product preparation module (not shown), where the product preparation module is arranged to receive and dilute the concentrated foamate of biosurfactant that is received from a plurality of holding tanks 150 using DI water. Any leftover mixture of foamate and aqueous biosurfactant remaining in the reactors may also be added to the product preparation module to dilute the concentrated foamate of biosurfactant. The concentrated foamate of biosurfactant is diluted in this manner to produce a diluted product for transport and sale. For example, the product may be diluted to a concentration of 50 ppm.

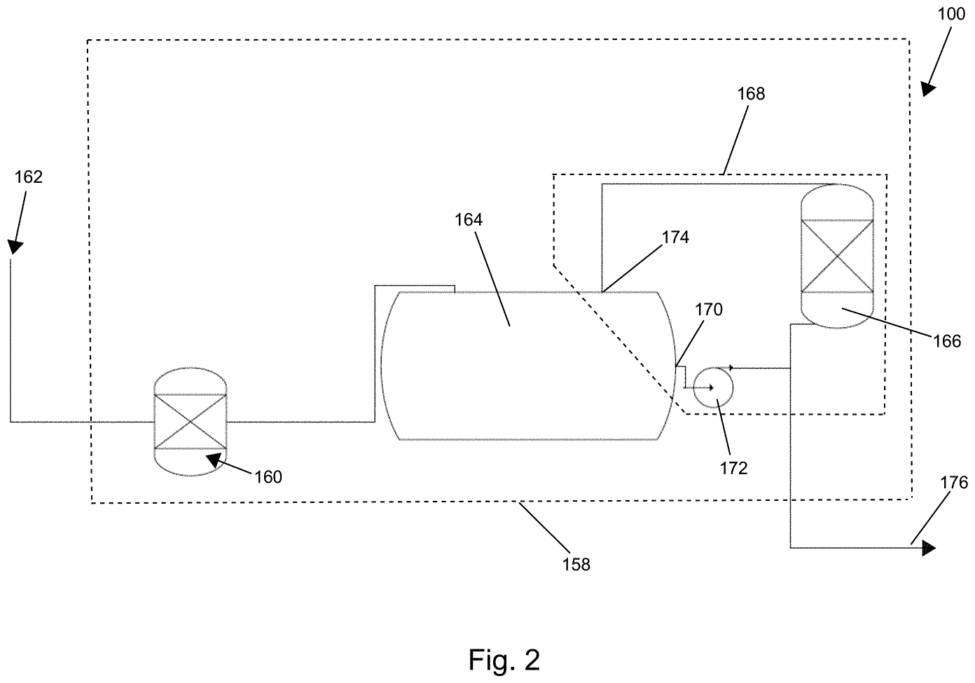

Referring now to FIG. 2, an embodiment is provided wherein, the system 100 further includes a purified water module 162 arranged to supply sterilized DI water to the reactors. The purified water module 162 may include a number of sub-modules. For example, the purified water module 162 may include a heating module (not shown) to heat the water from the water supply. For example, the growth of the at least one strain of Bacillus subtilis within the system 100 is intensified when the temperature of the mixture of foamate and aqueous biosurfactant is within the temperature range of 40 to 50° C. Therefore, when adding the water to the starter reactor 102, the water added may be heated by the heating module to a temperature range of 40 to 50° C. The heating module may include an in-tank or tank-type water heater, which includes at least one electrical resistance element within the tank surrounded by water. A current is passed through the element generating heat and heating the surrounding water.

In another embodiment, each of the reactors may also include their own heating systems to maintain the temperature range of 40 to 50° C. during fermentation, as maintaining the temperature of each of the reactors to within this range aids in concentrated foamate production. For example, each reactor may include a heating jacket (not shown) in series connection with a small water tank including an in-tank heater. The in-tank heater heats the water in the tank, where it is recirculated by a pump to and along the jacket and back into to the small tank. In a further embodiment, the mixture within each of the reactors may be heated by a pipe arranged within the reactors. Hot water may be pumped along the inside of the pipe, heating the mixture of foamate and aqueous biosurfactant surrounding the pipe. The hot water inside the pipe is heated within a small water tank including an in-tank heater.

However, the activity of the at least one strain of Bacillus subtilis in producing the biosurfactant generates heat. Therefore, the system 100 may further include features that prevent the temperature from exceeding 50° C. For example, each of the reactors may include cooling jackets. In an alternate embodiment, the system may include a temperature monitoring system (not shown) that is arranged to automatically monitor and maintain the mixture of foamate and aqueous biosurfactant to within the range of 40 to 50° C. That is, each reactor may include its own temperature monitoring system, where the temperature monitoring system may include a thermostat that periodically or continuously senses the temperature within each of the reactors. In response to the sensed temperature, the temperature monitoring system may then adjust the temperature of a reactor by heating or cooling the reactor by means of an adjustable heating/cooling jacket or by some other known means.

In another embodiment, the purified water module 162 may include a filtration module 160, which is arranged to receive water from a municipal water supply 162 and filter to remove particulates and minerals from the water supply to produce DI water. For example, the filtration module 160 may include an inline resin filter cartridge in connection with a water supply pipe. Once filtered, the DI water is collected and held in a water tank 164.

In a further embodiment, the purified water module 162 may include a sterilisation module 166 to sterilise the water from the water supply. The sterilisation module 166 may be connected in a recirculating loop 168 with the water tank 164 to ensure that any new DI water entering the tank is continuously sterilised before being used in the system 100. A recirculation outlet pipe 170 may be arranged in connection with a pump 172. The pump 172 may be arranged to pump the DI water around the recirculating loop 168 and through the sterilisation module 166. The sterilisation module 166 may use Ultra Violet (UV) radiation to sterilize the DI water. The sterilized DI water is pumped out of the sterilisation module 166 and back into the water tank 164 via the recirculation inlet pipe 174. Further, the system 100 may include a sterilized DI water inlet pipe 176 that is in connection with the rest of the system 100. That is, the purified water module 162 supplies sterilized DI water, which may be preheated, to the reactors for fermentation. The sterilized DI water may also be used in other parts of the process such as forming the inoculum or diluting the final product. The purified water module 162 ensures that no minerals or other bacteria enter the system 100 that might negatively affect the concentrated foamate production of the at least one strain of Bacillus subtilis.

In a further embodiment, there is provided a process for producing a biosurfactant at least one strain of Bacillus subtilis, using the above described system 100. The process may comprise the following steps. Firstly, the process may include the step of producing a mixture of foamate and aqueous biosurfactant cultured from the at least one strain of Bacillus subtilis. The mixture of foamate and aqueous biosurfactant may be then fermented in the at least one reactor to produce a concentrated foamate of biosurfactant. The at least one reactor may arranged to oxygenate the mixture of foamate and aqueous biosurfactant using the oxygenation module 110, agitate the mixture of foamate and aqueous biosurfactant using the agitation module, and recirculate the mixture of foamate and aqueous biosurfactant using the recirculation module 112, wherein the process further comprises the step of continuously collecting the concentrated foamate of biosurfactant from the at least one reactor using the collection module 114.

In a further embodiment, there is provided a method wherein the above method steps are repeated across different time periods within the system 100 so that the system 100 is continuously producing and collecting concentrated surfactant. For example, where the method is carried out for the first reactor 104 in a first time period, the second reactor 106 in a second time period, and the third reactor 108 in a third time period. That is, the commencement of fermentation is scattered across the different reactors. As would be understood by a person skilled in the art, the system 100 may need to be modified to ensure that each reactor remains a closed and separate system and cross-contamination of bacteria between reactors is minimised to reduce the risk of mutation. As such, additional pipes, pumps, and valves may be required to control the follow of the fluids and foamate around the system 100 that are not shown within the above figures.

In the foregoing description of preferred embodiments, specific terminology has been resorted to for the sake of clarity. However, the invention is not intended to be limited to the specific terms so selected, and it is to be understood that each specific term includes all technical equivalents, which operate in a similar manner to accomplish a similar technical purpose. Terms such as “front” and “rear”, “above” and “below” and the like are used as words of convenience to provide reference points and are not to be construed as limiting terms.

Optional embodiments may also be said to broadly include the parts, elements, steps and/or features referred to or indicated herein, individually or in any combination of two or more of the parts, elements, steps and/or features, and wherein specific integers are mentioned which have known equivalents in the art to which the invention relates, such known equivalents are deemed to be incorporated herein as if individually set forth.

Although a preferred embodiment has been described in detail, it should be understood that modifications, changes, substitutions or alterations would be apparent to those skilled in the art without departing from the scope of the present invention.

Throughout this specification and the claims which follow, unless the context requires otherwise, the word “comprise”, and variations such as “comprised”, “comprises” or “comprising”, will be understood to imply the inclusion of a stated integer or step or group of integers or steps but not the exclusion of any other integer or step or group of integers or steps.

As used herein, a, an, the, at least one, and one or more are used interchangeably, and refer to one or to more than one (i.e. at least one) of the grammatical object. By way of example, “an element” means one element, at least one element, or one or more elements.

In the context of this specification, the term “about” is understood to refer to a range of numbers that a person of skill in the art would consider equivalent to the recited value in the context of achieving the same function or result.

### Advantages

The embodiments described herein provide a novel system for producing a concentrated foamate of biosurfactant. Although, other known methods may be capable of producing the biosurfactant, this system produces the biosurfactant in a highly concentrated form in the foamate. The production of a concentrated form of the biosurfactant enables improved cost efficiencies during the manufacturing process, which is diluted to form a greater volume of product. That is, the embodiments of the system as described above enable a greater volume of biosurfactant produced compared to past systems. The increase in concentration of the biosurfactant derives from the synergistic relationship between the modules of the system. For instance, the described system does not require the use of a mechanical foam breaker within the foam fractionation column instead the column sends foam to the collection tank and because of the change in diameter between the pipe and the tank, this acts as foam breaker

Further, the embodiments of the described system enable a higher quality and more consistent end product as the product is diluted to the desired concentration. This also benefits the end user as the system produces a concentrated product that may be made into a number of different products at different concentrations to satisfy the market.

Furthermore, the system is in a modular way that enables effective scaling to suit demand. For example, the system may include one reactor or multiple reactors. The reactors may be of different sizes, but all reactors are aerated by a single oxygenation module and purified water supply module. Additionally, the modularity of the system enables it to be easily accommodated amongst existing processing plants or in challenging spaces.

Moreover, the system described is one that is safe and easily cleanable between operational periods. This reduces the risk to operators and to contamination of the system.

Furthermore, the embodiments described provide a system that effectively reduces contamination such that the addition of preservatives, such as Potassium sorbate and sodium benzoate, are no longer required to control the growth of mould and other complex organisms.

Formulations

Also provided herein are aqueous disinfectant formulations comprising:
(a) a biosurfactant composition comprising Surfactin, Iturin, Fengycin, and sodium dodecylbenzene sulfonate (DDBSA), wherein the Surfactin is present within the composition at about 25-75 ppm,
(b) thymol;
(c) lactic acid; and
(d) preservative;
   wherein the formulations have a pH in the range of about 2.5-4.5.

In an embodiment the disinfectant formulation is in the form of a sprayable, fine mist or foggable liquid.

In an embodiment the disinfectant formulation is present in a disinfectant wipe.

In a further aspect the invention provides a method of disinfecting a substrate surface comprising applying to said surface an effective amount aqueous disinfectant composition comprising:

(a) biosurfactant composition comprising Surfactin, Iturin, Fengycin, and sodium dodecylbenzene sulfonate (DDBSA), wherein the Surfactin is present within the composition at between about 25-75 ppm,
(b) thymol;
(c) lactic acid; and
(d) preservative;
wherein the formulation has a pH in the range of about 2.5-4.5.

In an embodiment the method is conducted for the purpose of disinfecting a surface, a drain, or wastewater inlet against a bacteria, algae, fungi, mould, and/or virus.

In an embodiment the bacteria, fungi, and/or virus is selected from the group consisting of Staphylococcus aureus (including MRSA), Escherichia coli (E. coli), Pseudomonas, Proteus vulgaris, Salmonella choleraesuis, Clostridium difficile, Enterococcus (including Vancomycin-resistant enterococci (VRE)), human coronavirus, Influenza, and Stachybotrys chartarum.

In an embodiment the method is conducted for the purpose of disinfecting a surface a drain, or wastewater inlet against a bacteria, and in particular Staphylococcus aureus or MRSA.

In an embodiment the method is conducted for the purpose of disinfecting a surface a drain, or wastewater inlet against a bacteria, and in particular E. coli.

In an embodiment the method is conducted for the purpose of disinfecting a surface against a virus, and in particular human coronavirus.

In an embodiment the method is conducted for the purpose of disinfecting a surface a drain, or wastewater source against human coronavirus, and in particular the strain SARS-CoV-2 which causes the COVID-19 disease state.

As used herein the term “disinfectant” refers to a substance that is applied to a non-living/non-biological object (and in particular, a substrate surface) to destroy microorganisms or viruses that may be present on the object. In the context of the present invention the formulations comprises amounts of multiple antibacterial, antifungal, and antiviral agents which together display synergistic or supra additive effects. It will be appreciated that in the context of the present invention the term “disinfectant” may also encompass the concept of sanitization, as the compositions of the present invention may also serve to disinfect and clean. Without being bound to any particular mode of action the formulations of the present invention may also, in some embodiments, be classed as biocides in the context of being able to destroy viruses, in addition to microorganism such as bacteria. In relation to this latter embodiment the formulations may be thought as antibacterial disinfectants.

It will be appreciated that an “effective amount” as used herein refers to an amount of the formulation which is applied to a surface to disinfect the surface against viruses (ex vivo), bacteria, or fungi. Disinfection is readily achieved where the number of microorganisms killed is a Log reduction of at least 4.0 which means that less than 1 microorganism in 10,000 remains. The formulations of the present invention may provide Log reductions of at least 4.0, preferably at least 5.0, and more preferably at least about 6.0.

The biosurfactant composition within the formulation comprises Surfactin, Iturin, Fengycin, and sodium dodecylbenzene sulfonate (DDBSA), wherein the Surfactin is present within the composition at about 25-75 ppm. The skilled person will understand that this composition can be produced by the method and system disclosed herein. The level of Surfactin may vary between about 25-75 ppm. The DDBSA is also a byproduct of the method and system disclosed herein and is generally present in the total aqueous composition in a range of from about 0.3 to about 3% v/v, such as 0.35, 0.40, 0.45, 0.50 0.55, 0.60, 0.75, 0.80, 0.85, 0.95, 1.0, 1.2, 1.4, 1.6, 1.8, 2.0, 2.2, 2.4, 2.6, 2.8, and 2.9% v/v or any range between any two of these amounts.

“Sodium DDBSA” as used herein refers to sodium dodecylbenzene sulfonate which is a synthetically derived basic surface active agent.

In an embodiment the formulation comprises lactic acid total in a range of from about 0.3 to about 3% v/v, such as 0.35, 0.40, 0.45, 0.50 0.55, 0.60, 0.75, 0.80, 0.85, 0.95, 1.0, 1.2, 1.4, 1.6, 1.8, 2.0, 2.2, 2.4, 2.6, 2.8, and 2.9% v/v or any range between any two of these amount relative to the total of the volume of the aqueous disinfectant formulation; or at a concentration of about 3,000 to about 30,000 ppm, such as about 5,000, 7,000, 9,000, 10,000, 12,000, 15,000, 17,000, 19,000, 21,000, 23,000, 25,000, 27,000, 29,000 ppm or any range between any two of these concentrations.

In an embodiment the formulation comprises thymol total in a range of from about 0.005 to about 0.5% v/v, such as 0.007, 0.009, 0.01, 0.015, 0.02, 0.25, 0.03, 0.035, 0.040, 0.045, 0.050, 0.055, 0.060, 0.065, 0.070, 0.075, 0.080, 0.085, 0.090, 0.095, 0.10, 0.15, 0.20, 0.25, 0.30, 0.35, 0.40, and 0.45% v/v or any range between any two of these amounts relative to the total of the volume of the aqueous disinfectant formulation; or at a concentration of about 50 to about 5,000 ppm, such as about 55, 65, 80, 90, 100, 150, 200, 250, 300, 500, 600, 700, 800, 900, 1000, 1200, 1500, 1700, 1900, 2300, 2500, 3000, 3200, 3600, 3800, 4200, 4400, 4600, 4800 ppm or any range between any two of these concentrations.

In an embodiment the invention provides are aqueous disinfectant formulations comprising:
(a) a biosurfactant composition comprising Surfactin, Iturin, Fengycin, and sodium dodecylbenzene sulfonate (DDBSA), wherein the Surfactin is present within the composition at about 25-75 ppm, wherein the DDBSA is present in the formulation at about 0.3%-3% v/v;
(b) thymol which is present in the formulation at about 0.3%-3% v/v;
(c) lactic acid which is present in the formulation at about 0.005%-0.5% v/v; and
(d) preservative which is present in the formulation at about 0.05%-0.5% v/v;
wherein the formulations have a pH in the range of about 2.5-4.5.

In an embodiment the invention provides are aqueous disinfectant formulations comprising:
(a) a biosurfactant composition comprising Surfactin, Iturin, Fengycin, and sodium dodecylbenzene sulfonate (DDBSA), wherein the Surfactin is present within the composition at about 25-75 ppm, wherein the DDBSA is present in the formulation at about 1% v/v.
(b) thymol which is present in the formulation at about 0.06% v/v;
(c) lactic acid which is present in the formulation at about 1% v/v; and
(d) preservative which is present in the formulation at about 0.3% v/v;
wherein the formulations have a pH in the range of about 2.5-4.5.

In an embodiment the preservative is selected from sodium benzoate or potassium sorbate.

Accordingly, the invention also provides are aqueous disinfectant formulations comprising:
(a) biosurfactant composition comprising Surfactin, Iturin, Fengycin, and sodium dodecylbenzene sulfonate (DDBSA), wherein the Surfactin is present within the composition at about 25-75 ppm, wherein the DDBSA is present in the formulation at about 1% v/v.
(b) thymol which is present in the formulation at about 0.06% v/v;
(c) lactic acid which is present in the formulation at about 1% v/v; and
(d) preservative which is present in the formulation at about 0.3% v/v, wherein the preservative is selected from sodium benzoate or potassium sorbate;
wherein the formulations have a pH in the range of about 2.5-4.5.

Thymol is found in the oil of thyme and is extracted from the plant Thymus vulgaris. It is a naturally occurring phenol and plant derived. In an embodiment the thymol is thymol oil available from ParChem or Sigma Aldrich. Thymol oil has a viscosity (measured at 25° C.) of from about 80,000-900,000 cps, for instance, about 90,000-800,000 cps or about 100,000-700,000 cps.

In an embodiment the aqueous disinfectant formulation comprises about 70-90% v/v of water, such as about 71%, 72%, 74%, 76%, 78%, 80%, 82%, 84%, 86%, 88%, or about 89% or any range between two of these figures.

The disinfectant formulation of the present invention may also include additional ingredients such as acids (e.g., hydrochloric acid, sulphuric acid, etc); bases (e.g., sodium hydroxide, sodium carbonate, etc); other surfactants (e.g., labs acid/laurylbenzene sulfonic acid, CTAB, cocodiethanolamide (CDE, or CD80), SLES or sodium laureth sulfate, soap noodles, glycols, etc); other disinfecting agents (e.g., formaldehyde (or other aldehydes), ethanol or isopropyl alcohol (or other alcohols), sodium hypochlorite (or other hypochlorites), glycols, chloroamine, hydrogen peroxide, chlorine dioxide, permanganates, peracetic acid, performic acid, phenol (and other phenolics), and quartenary ammonium compounds such as benzalkonium chloride, etc); fragrances; antioxidants; phosphates (e.g., sodium tripolyphosphate (STPP)) and colouring agents.

In an embodiment the formulations of the present invention may also be characterized with traces of disodium phosphate.

In an embodiment the formulations of the present invention are chlorine free. For instance the compositions of the invention do not include sodium hypochlorite (or other hypochlorites), chloroamine, chlorine dioxide, and the like.

In an embodiment the formulations comprise traces of disodium phosphate and chlorine free.

In an embodiment any additional components in the specification do not constitute more than 15% wt/wt, of the total disinfectant composition. Typically, when present, the additional components comprise between about 2-10% wt/wt of the total disinfectant composition, such as about 2-4%, 2-6%, or 4-8%. wt/wt of the total disinfectant composition.

The pH of the disinfectant composition is 2.5-4.5, more preferably 3-4, and most preferably about 3.5.

In an embodiment the disinfectant formulation is in the form of a sprayable liquid which may be applied to a substrate by way of a hand-actuated or pressurised spray delivery device (e.g., spray gun). In this regard, it is preferable that the viscosity of the formulation in the form of a sprayable liquid is from 1 to 5 cps (measured at 25° C.).

In another embodiment the disinfectant formulation may be first absorbed by an applicator device (e.g., mob, cloth, cotton bud, paint brush, etc) and applied to a substrate.

In an embodiment the disinfectant formulation is provided in the form of a disinfectant wipe.

The wipe may improve formulation performance by providing mechanical/physical cleaning properties. The wipes of the invention comprise an absorbent substrate, for instance, an absorbent nonwoven water insoluble substrate, which has been impregnated with the disinfectant formulation. The wipe may take the form of a towellette, cloth, sheet, pad, or sponge and may also be associated with a holder device or applicator device such as a handle. The impregnation step involves contacting the wipe with the formulation, for instance, by spraying or immersing the wipe with the composition for a time and under conditions sufficient to allow for the wipe to be impregnated with the formulation.

In an embodiment the wipe is a nonwoven water insoluble material (substrate) which is synthetic or of plant origin. Such materials include rayon, polyester, nylon, polyethylene, cotton, or cardboard.

The substrate for the wipes may be impregnated with the disinfecting formulation at the loading level from about 1.5 times the original weight of the wipe to about 10 times the original weight of the wipe, preferably from about 2.5 times to about 7.5 times, and more preferably from about 3 times to about 6 times.

The formulation of the present invention may be applied to any substrate which may come into contact with a microorganism or virus, such as in a hospital setting. Accordingly, contemplated substrates include plastics/polymer surfaces (e.g., polyesters, PVC, etc), stainless steel, wood, glass, laminates, ceramic, and so on.

In relation to the disinfectant qualities the present formulation may be suitable for disinfecting a surface against the following: methicillin resistant Staphylococcus aureus (including MRSA), Staphylococcus aureus, human coronavirus, influenza A, Listeria monocyto genus, herpes simplex virus type 1, Escherichia coli (E. coli), Acinetobacter baumannii, vancomycin resistant Enterococcus faecium (VRE), Bacillus cereus, Klebsiella pneumoniae, rotavirus, human immunodeficient virus type 1, Pseudomonas aeruginosa, norovirus, Salmonella choteraesuis, Clostridium difficile, rhinovirus, and Trichophyton mentagrophytes (Athlete's foot fungi) and Stachybotrys chartarum (Toxic Black Mould).

In an embodiment the bacteria, algae, mould, fungi, and/or virus is selected from the group consisting of Staphylococcus aureus (including MRSA), Escherichia coli (E. coli), Pseudomonas, Proteus vulgaris, Salmonella choleraesuis, Clostridium difficile, and Enterococcus (including Vancomycin-resistant enterococci (VRE)).

Preferably the disinfectant qualities of the formulations are suitable for disinfecting a surface against a gram-positive bacteria, preferably clostridium, Enterococcus, or Staphylococcus.

Preferably the disinfectant qualities of the composition are suitable for disinfecting a surface against a gram-negative bacteria, preferably Escherichia, Pseudomonas, Proteus vulgaris, and Salmonella.

Preferably the disinfectant qualities of the composition are suitable for disinfecting a surface against a bacteria, and preferably Staphylococcus aureus and MRSA.

In an embodiment the formulation provides a Log reduction of at least 4.0 for 24-48 hrs.

In an embodiment the formulation provides a Log reduction of at least 4.0 for about 48 hrs.

In an embodiment the formulation provides a Log reduction of at least 4.0 for about 48-72 hrs.

In another embodiment the formulation provides a Log reduction of at least 4.0 for 24-72 hrs.

In a further embodiment the formulation provides a Log reduction of at least 4.0 for 24-96 hrs.

In a further embodiment the formulation provides a Log reduction of at least 4.0 for 24-120 hrs.

In addition to disinfecting surfaces, one of the other advantages of the formulation is its ability to remove grease and disinfect drains and the liquid within a drain trap. That is, in addition to the disinfecting qualities of the formulation, the formulation also has grease/oil solubilising properties and hence this unique dual purpose.

With the emergence of information about pathogen atomising and spreading from drains, it is becoming more important to disinfect drains. For instance the food industry has been attempting to stop atomised Listeria spreading with the use of peracetic acid treatments of drains. The present invention provides a beneficial alternative.

There is also concern starting to emerge around vector transmission of pathogens from drains, in particular “drain” or “bar” flies transmitting pathogens such as Listeria from drains onto food contact surfaces.

Accordingly, in another aspect the invention provides a method for minimising the transmission of pathogens from drains, including the step of adding to said drain, and effective amount aqueous disinfectant formulation comprising:

(a) biosurfactant composition comprising Surfactin, Iturin, Fengycin, and sodium dodecylbenzene sulfonate (DDBSA), wherein the Surfactin is present within the composition at between about 25-75 ppm,
(b) thymol;
(c) lactic acid; and
(d) preservative;
wherein the formulation has a pH in the range of about 2.5-4.5.

Accordingly, in another aspect the invention provides a method for minimising the transmission of pathogens from drains while also at the same time removing grease, including the step of adding to said drain, and effective amount aqueous disinfectant composition comprising:

(a) biosurfactant composition comprising Surfactin, Iturin, Fengycin, and sodium dodecylbenzene sulfonate (DDBSA), wherein the Surfactin is present within the composition at between about 25-75 ppm,
(b) thymol;
(c) lactic acid; and
(d) preservative;
wherein the formulation has a pH in the range of about 2.5-4.5.
The invention will now be further explained by the following non-limiting examples.

The invention will now be further explained by the following non-limiting examples.

### EXAMPLES

Test Formulation

Results:

BioProtect Sanitizer w/Thymol Formula

| Surfactant | | DDBSA | | Preservatives | | Lactic Acid | | Thymol | |
|---|---|---|---|---|---|---|---|---|---|
| 50 | 0.005% | 10,000 | 1.000% | 0.300% | 3000 | 10,000 | 1.000% | 600 | 0.060% |
| ppm | v/v | ppm | v/v | v/v | ppm | ppm | v/v | ppm | v/v |

### Example 1

### Exposure Results

| | Pre- | Post- | |
|---|---|---|---|
| | Exposure | Exposure | Percent |
| Bacteria | Count | Count | Inhibited |
| Pseudomonas | 7.50e+05 | 2.25e+01 | 99.99% |
| aeruginosa | 75,000,000 cfu/mL | 2,250 cfu/mL | |
| Staphylococcus | 1.50e+05 | 1.25e+01 | 99.99% |
| aureus | 15,000,000 cfu/mL | 1,250 cfu/mL | |
| Escherichia | 5.50e+05 | 1.60e+01 | 99.99% |
| coli | 55,000,000 cfu/mL | 1,600 cfu/mL | |

### Analysis & Discussion:

This formulation has reduced the number of live organisms >99.99% on all species tested.
Internal testing shows that the product will inhibit Gram (+) & Gram (−) to acceptable limits.

### Methods

The formulation was evaluated in accordance with the TGA (Therapeutics Goods Act) Test for Commercial Grade Disinfectants (Option C). The tests were performed in triplicate utilizing fresh cultures and solutions on each occasion with the following results:

| | | | | |
|---|---|---|---|---|
| | | | Growth in | |
| | | Count | Recovery | |
| Test | Dilution | (Orgs/mL) | Broths | Results |

A. Escherichia coli NCTC 8196

| | | | | |
|---|---|---|---|---|
| 1 | Neat | 7.7 × 10⁸ | — | Pass |
| 3 | Neat | 1.6 × 10⁹ | — | Pass |
| 3 | Neat | 9.8 × 10⁸ | — | Pass |

B. Staphylococcus aurens NCTC 4163

| | | | | |
|---|---|---|---|---|
| 1 | Neat | 8.7 × 10⁸ | — | Pass |
| 2 | Neat | 7.1 × 10⁸ | — | Pass |
| 3 | Neat | 8.2 × 10⁸ | — | Pass |

Notes:
1. ‘—’ indicates no growth in recovery broths
2. ‘+’ indicates growth in recovery broths

The sample tested was found to pass the test under the above test conditions. All controls conformed to the requirements of the test procedure.

### Example 2

### Test Formulation Against Coronavirus (Murine Hepatitis Virus)

Eurofins Sample Number NJ20AA9611-1

| | |
|---|---|
| Original Received Date: | Jul. 24, 2020 |
| Description: | BioProtect Microbial Sanitiser |
| | 0.5 L; Exp: June 2022 |
| Lot Number: | 200720 |
| Containers Submitted: | 1 Bottle(s) |

Analysis
Virucidal Test by Carrier Method
Refer to Attachment #1
Method: TMCV 006, ASTM E1053
Analysis Date: Aug. 5, 2020
Contracted Company: Eurofins ams Laboratories Sydney
8. Rachael Close, Silverwater, NSW 2128 Australia
amslabs@eurofins.com
CONDITIONS

| | |
|---|---|
| Virus Strain | Murine hepatitis virus (MHV)-1 ATCC/R-261 |
| Cell Substrate | A9 cells ATCC/CCL-1.4 |
| Test Concentration | Neat |
| Contact Time | 10 minutes |
| Test Temperature | Room temperature |
| Test Condition | Dirty 5% FBS (Fetal Bovine Serum) |
| Neutraliser | 2% FBS in MM |

RESULTS: TABLE 1: MHV-1 test/control results for 10 minifies contact

| | Humber of | | | | |
|---|---|---|---|---|---|
| Virus | Inoculated | Virus | | | Test |
| Dilution | Wells | Control | Cytotoxicity | Neutralisation | Sample |
| 10⁻¹ | 4 | 4⁺/4 | C | C | C |
| 10⁻² | 4 | 4⁺/4 | C | C | C |
| 10⁻³ | 4 | 4⁺/4 | 0⁺/4 | 4⁺/4 | 1⁺/4 |
| 10⁻⁴ | 4 | 4⁺/4 | N/A | N/A | 1⁺/4 |
| 10⁻⁵ | 4 | 4⁺/4 | N/A | N/A | 0⁺/4 |
| 10⁻⁶ | 4 | 3⁺/4 | N/A | N/A | 0⁺/4 |
| 10⁻⁷ | 4 | 2⁺/4 | N/A | N/A | N/A |
| 10⁻⁸ | 4 | 2⁺/4 | N/A | N/A | N/A |
| Log₁₀ | — | 7.25 | 2.5 | 2.5 | 2.83 |

| | |
|---|---|
| Log₁₀ Reduction of Virus after Treatment | 4.42 |

Note:
Presence of virus in each response is recorded as “+”
Absence of virus in each response is recorded as “0”
Cytotoxic response is recorded as “C”
Calculated virus titre = 10^{7.25} TCID_{50/0.1 ml}(7.25 log₁₀)
Cell control-4 wells with healthy cell monolayer
* The Reed & Muench LD50 Method was used for determining the virus titre endpoint.

### Conclusions:

Considering the cytoxicity and neutralisation test results, the sample has shown virucidal efficacy against MHV-1 by achieving 4.42 log reduction in virus concentration after 10 minutes exposure period at room temperature.

### Example 3

A sample marked ‘BioProtect Microbial Sanitiser #200720’ was evaluated by the AOAC Hard Surface Carrier Test 991.47, 48, 49 (modified) under the following test conditions.
Product Dilution: Neat
Contact Time: 60 minutes
Soil: 5% Horse Serum
Diluent: Standard Hard Water

### Results

| | No. of | No. of | No. of |
|---|---|---|---|
| | Carriers | Carriers | Carriers |
| Test Organism | Tested | Negative | Positive |
| Pseudomonas aeruginosa | 60 | 60 | 0 |
| ATCC 15442 | | | |
| Staphylococcus aureus | 60 | 60 | 0 |
| ATCC 6538 | | | |
| Salmonella choleraesuis | 60 | 60 | 0 |
| ATCC 10708 | | | |

Notes
1. Test cultures and controls conformed to the requirements of the test.
2. According to AOAC 991.47, 48, 49, the disinfectant passes if ≤2 positive carriers out of 60 tested against S. aureus and Salmonella or ≤3 positive carriers out of 60 tested against P. aeruginosa is obtained.

### CONCLUSION

The product passed the test under the above prescribed conditions.

1. A system for producing a biosurfactant from at least one strain of Bacillus subtilis, the system comprising:
at least one reactor arranged to receive a mixture of foamate and aqueous biosurfactant cultured from the at least one strain of Bacillus subtilis, the at least one reactor being arranged to ferment the mixture of foamate and aqueous biosurfactant into a concentrated foamate of biosurfactant, the at least one reactor comprising:
an oxygenation module arranged to oxygenate the mixture of foamate and aqueous biosurfactant;
an agitation module arranged to agitate the mixture of foamate and aqueous biosurfactant;
a recirculation module arranged to recirculate the mixture of foamate and aqueous biosurfactant; and
the system further including a collection module in connection with the at least one reactor that is arranged to continuously collect the concentrated foamate of biosurfactant.

2. The system in accordance with claim 1 wherein the at least one strain of Bacillus. subtilis is strain RSA-203 deposited with the ATCC on 9 Jan. 2013 under Accession No. PTA-13451.

3. The system in accordance with claim 1, wherein the at least one reactor is a cylindrical vessel, with a base, a top and a side wall.

4. The system in accordance with claim 3, wherein, the at least one reactor has a height to a diameter ratio of 3:1.

5. The system in accordance with claim 1 to 11, wherein the recirculation module includes:
a draining pipe in connection with at least one drain formed in the base or the side wall of the at least one reactor;
a pump arranged to pump the mixture of foamate and aqueous biosurfactant drained into the draining pipe;
a recirculation tank in connection with the draining pipe, the recirculation tank arranged to include at least one recirculation air diffuser to oxygenate the mixture of foamate and aqueous biosurfactant in the recirculation tank; and
a recirculation pipe arranged reintroduce the mixture of foamate and aqueous biosurfactant from the recirculation tank into the at least one reactor.

6. The system in accordance with claim 1, wherein the agitation module includes at least one agitation mechanism arranged to generate a flow path in the mixture of foamate and aqueous biosurfactant.

7. The system in accordance with claim 6, wherein the at least one agitation mechanism includes an impeller connected to a rotation shaft located at the base of the at least one reactor.

8. The system in accordance with claim 6, the agitation module further includes at least one baffle arranged to extend along at least partially the side of wall between the base and the top.

9. The system in accordance with claim 7, wherein the flow path is a substantially circular path around a circumference of the at least one reactor.

10. The system in accordance with claim 7, wherein the flow path is a substantially conical helix shape within the at least one reactor.

11. The system in accordance with claim 1, wherein the concentration of the concentrated foamate of biosurfactant is in the range of 50 to 2000 ppm.

12. The system in accordance with claim 1, wherein the oxygenation module includes at least one air diffuser arranged within the at least one reactor to supply air to the mixture of foamate and aqueous biosurfactant.

13. The system in accordance with claim 12, wherein the air includes a mix of pure oxygen and pressurised air.

14-31. (canceled)

32. A process for producing a biosurfactant from at least one strain of Bacillus subtilis, the process comprising the following steps:
producing a mixture of foamate and aqueous biosurfactant cultured from the at least one strain of Bacillus subtilis,
fermenting the mixture of foamate and aqueous biosurfactant in at least one reactor to produce a concentrated foamate of biosurfactant, the at least one reactor arranged to:
oxygenate the mixture of foamate and aqueous biosurfactant using an oxygenation module;
agitate the mixture of foamate and aqueous biosurfactant using an agitation module; and
recirculate the mixture of foamate and aqueous biosurfactant using a recirculation module;
wherein the process further comprises the step of continuously collecting the concentrated foamate of biosurfactant from the at least one reactor using a collection module.

33. A process in accordance with claim 32 wherein the at least one strain of Bacillus. subtilis is strain RSA-203 deposited with the ATCC on 9 Jan. 2013 under Accession No. PTA-13451.

34. An aqueous disinfectant formulation comprising:
(a) biosurfactant composition comprising Surfactin, Iturin, Fengycin, and sodium dodecylbenzene sulfonate (DDBSA), wherein the Surfactin is present within the composition at about 25-75 ppm,
(b) thymol;
(c) lactic acid; and
(d) preservative;
wherein the formulations have a pH in the range of about 2.5-4.5.

35-36. (canceled)

37. A method of disinfecting a substrate surface comprising applying to said surface an effective amount aqueous disinfectant formulation according to claim 34.

38. A method according to claim 37 for disinfecting a surface, a drain, or wastewater inlet against a bacteria, algae, fungi, mould, and/or virus.

39-41. (canceled)

42. An aqueous disinfectant formulation according to claim 34 comprising:
(a) biosurfactant composition comprising Surfactin, Iturin, Fengycin, and sodium dodecylbenzene sulfonate (DDBSA), wherein the Surfactin is present within the composition at about 25-75 ppm, wherein the DDBSA is present in the formulation at about 0.3%-3% v/v.
(b) thymol which is present in the formulation at about 0.3%-3% v/v;
(c) lactic acid which is present in the formulation at about 0.005%-0.5% v/v; and
(d) preservative which is present in the formulation at about 0.05%-0.5% v/v;
wherein the formulations have a pH in the range of about 2.5-4.5.

43. (canceled)

44. A method for minimising the transmission of pathogens from drains, including the step of adding to said drain, and effective amount of an aqueous disinfectant formulation according to claim 34.

45. (canceled)
